(19) United States
(12) Patent Application Publication
(10) Pub. No.: US 2007/0265259 A1
(43) Pub. Date: Nov. 15, 2007

(54) Proline Derivatives

(76) Inventors: Werner Mederski, Zwingenberg (DE); Christos Tsaklakidis, Weinheim (DE); Dieter Dorsch, Ober-Ramstadt (DE); Bertram Cezanne, Moerfelden-Walldorf (DE); Johannes Gleitz, Darmstadt (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/576,226

(22) PCT Filed: Sep. 16, 2005
(86) PCT No.: PCT/EP05/10025

§ 371 Date: Mar. 28, 2007

(30) Foreign Application Priority Data
Sep. 29, 2004 (DE) 10 2004 047 255.6

(51) Int. Cl. A61K 31/5377; A61P 29/00; A61P 35/00; A61P 35/04; A61P 9/00; C07D 413/14

(52) U.S. Cl. 514/234.2; 544/141

(57) ABSTRACT

Novel compounds of the formula (I), in which X, Y, R¹, R², R³, R⁴ and n have the meaning indicated in Patent Claim 1, are inhibitors of coagulation factor Xa and can be employed for the prophylaxis and/or therapy of thromboembolic diseases and for the treatment of tumours.

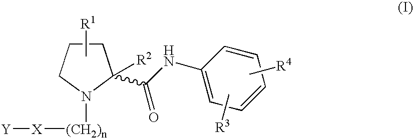

The invention relates to compounds of the formula I 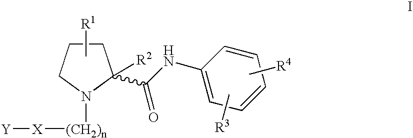
in which
R¹ denotes H, ═O, Hal, A, OH, OA, —O—(CH₂)ₘ—OA, A-COO—, Ph-(CH₂)ₙ—COO—, cycloalkyl-(CH₂)ₙ—COO—, A-CONH—, A-CONA-, Ph-CONA-, N₃, NH₂, NO₂, CN, COOH, COOA, CONH₂, CONHA, CON(A)₂, O-allyl, O-propargyl, O-benzyl, ═N—OH, ═N-OA or ═CF₂,
R² denotes H or A,
Ph denotes phenyl which is unsubstituted or mono-, di- or trisubstituted by A, OA, OH and/or Hal,
R³ denotes H, Hal or A,
R⁴ denotes 2-oxopiperidin-1-yl, 2-oxopyrrolidin-1-yl, 2-oxo-1H-pyridin-1-yl, 3-oxomorpholin-4-yl, 4-oxo-1H-pyridin-1-yl, 2-oxo-1H-pyrazin-1-yl, 2-oxoimidazolidin-1-yl, 2-iminopiperidin-1-yl, 2-iminopyrrolidin-1-yl, 3-iminomorpholin-4-yl, 2-iminoimidazolidin-1-yl, 2-imino-1H-pyrazin-1-yl, 2,6-dioxo-piperidin1-yl, 2-oxopiperazin-1-yl, 2,6-dioxopiperazin-1-yl, 2,5-dioxopyrrolidin-1-yl, 2-oxo-1,3-oxazolidin-3-yl, 3-oxo-2H-pyridazin-2-yl, 2-caprolactam-1-yl (=2-oxoazepan-1-yl), 2-azabicyclo[2.2.2]-octan-3-on-2-yl, 5,6-dihydro-1H-pyrimidin-2-oxo-1-yl, 2-oxo-1,3-oxazinan-3-yl or 4H-1,4-oxazin-4-yl, optionally mono- or disubstituted by A, OA, OH and/or CN,
X denotes a bond, CONH or NHCO,
Y denotes phenyl, pyridyl, thienyl, pyrimidyl, benzo[b]thiophenyl, each of which is unsubstituted or mono-, di-, tri-o tetra- or penta-substituted by Hal,
   or Z,
Z denotes 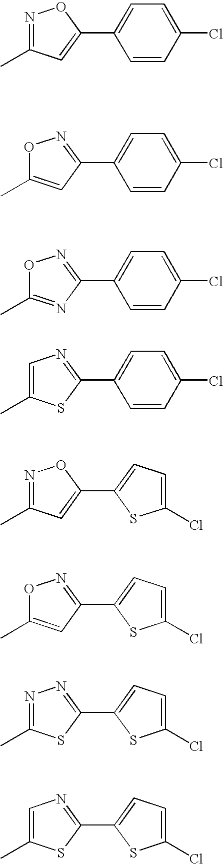
A denotes unbranched, branched or cyclic alkyl having 1-10 C atoms, in which, in addition, 1-7H atoms may be replaced by F and/or chlorine,
Hal denotes F, Cl, Br or I
n denotes 1 or 2,
   and pharmaceutically usable derivatives, solvates, salts and stereoisomers thereof, including mixtures thereof in all ratios.

The invention had the object of finding novel compounds having valuable properties, in particular those which can be used for the preparation of medicaments.

It has been found that the compounds of the formula I and salts thereof have very valuable pharmacological properties while being well tolerated.

In particular, they exhibit factor Xa-inhibiting properties and can therefore be employed for combating and preventing thromboembolic diseases, such as thrombosis, myocardial infarction, arteriosclerosis, inflammation, apoplexy, angina pectoris, restenosis after angioplasty and claudicatio intermittens.

The compounds of the formula I according to the invention may further-more be inhibitors of the coagulation factors factor VIIa, factor IXa and thrombin in the blood coagulation cascade.

Other carboxamide derivatives are disclosed in WO 02/48099 and WO 02/57236, other pyrrolidine derivatives are described in WO 02/100830.

Further heterocyclic derivatives are disclosed in WO 03/045912, WO 2004/056815, and by M. Nazare et al Bioorg. Med. Chem. Lett. 2004, 14, 4192 and by M. Nazaré et al. Bioorg. Med. Chem. Lett. 2004, 14, 4197.

The antithrombotic and anticoagulant effect of the compounds according to the invention is attributed to the inhibitory action against activated co-agulation protease, known by the name factor Xa, or to the inhibition of other activated serine proteases, such as factor VIIa, factor IXa or thrombin.

Factor Xa is one of the proteases involved in the complex process of blood coagulation. Factor Xa catalyses the conversion of prothrombin into thrombin. Thrombin cleaves fibrinogen into fibrin monomers, which, after cross-linking, make an elementary contribution to thrombus formation. Activation of thrombin may result in the occurrence of thromboembolic diseases. However, inhibition of thrombin may inhibit the fibrin formation involved in thrombus formation.

The inhibition of thrombin can be measured, for example by the method of G. F. Cousins et al. in Circulation 1996, 94, 1705-1712.

Inhibition of factor Xa can thus prevent the formation of thrombin. The compounds of the formula I according to the invention and salts thereof engage in the blood coagulation process by inhibiting factor Xa and thus inhibit the formation of thrombuses.

The inhibition of factor Xa by the compounds according to the invention and the measurement of the anticoagulant and antithrombotic activity can be determined by conventional in-vitro or in-vivo methods. A suitable method is described, for example, by J. Hauptmann et al. in Thrombosis and Haemostasis 1990, 63, 220-223.

The inhibition of factor Xa can be measured, for example by the method of T. Hara et al. in Thromb. Haemostas. 1994, 71, 314-319.

Coagulation factor VIIa initiates the extrinsic part of the coagulation cascade after binding to tissue factor and contributes to the activation of factor X to give factor Xa. Inhibition of factor VIIa thus prevents the formation of factor Xa and thus subsequent thrombin formation.

The inhibition of factor VIIa by the compounds according to the invention and the measurement of the anticoagulant and antithrombotic activity can be determined by conventional in-vitro or in-vivo methods. A conventional method for the measurement of the inhibition of factor VIIa is described, for example, by H. F. Ronning et al. in Thrombosis Research 1996, 84, 73-81.

Coagulation factor IXa is generated in the intrinsic coagulation cascade and is likewise involved in the activation of factor X to give factor Xa. Inhibition of factor IXa can therefore prevent the formation of factor Xa in a different way.

The inhibition of factor IXa by the compounds according to the invention and the measurement of the anticoagulant and antithrombotic activity can be determined by conventional in-vitro or in-vivo methods. A suitable method is described, for example, by J. Chang et al. in Journal of Biological Chemistry 1998, 273, 12089-12094.

The compounds according to the invention may furthermore be used for the treatment of tumours, tumour diseases and/or tumour metastases. A correlation between tissue factor TF/factor VIIa and the development of various types of cancer has been indicated by T. Taniguchi and N. R. Lemoine in Biomed. Health Res. (2000), 41 (Molecular Pathogenesis of Pancreatic Cancer), 57-59.

The publications listed below describe an antitumoural action of TF-VII and factor Xa inhibitors for various types of tumour:

K. M. Donnelly et al. in Thromb. Haemost. 1998; 79: 1041-1047;

E. G. Fischer et al. in J. Clin. Invest. 104: 1213-1221 (1999);

B. M. Mueller et al. in J. Clin. Invest. 101: 1372-1378 (1998);

M. E. Bromberg et al. in Thromb. Haemost. 1999; 82: 88-92

The compounds of the formula I can be employed as medicament active ingredients in human and veterinary medicine, in particular for the treatment and prevention of thromboembolic diseases, such as thrombosis, myocardial infarction, arteriosclerosis, inflammation, apoplexy, angina pectoris, restenosis after angioplasty, claudicatio intermittens, venous thrombosis, pulmonary embolism, arterial thrombosis, myocardial ischaemia, unstable angina and strokes based on thrombosis.

The compounds according to the invention are also employed for the treatment or prophylaxis of arteriosclerotic diseases, such as coronary arterial disease, cerebral arterial disease or peripheral arterial disease.

The compounds are also employed in combination with other thrombolytic agents in myocardial infarction, furthermore for prophylaxis for reocclusion after thrombolysis, percutaneous transluminal angioplasty (PTCA) and coronary bypass operations.

The compounds according to the invention are furthermore used for the prevention of rethrombosis in microsurgery, furthermore as anticoagulants in connection with artificial organs or in haemodialysis.

The compounds are furthermore used in the cleaning of catheters and medical aids in patients in vivo, or as anticoagulants for the preservation of blood, plasma and other blood products in vitro. The compounds according to the invention are furthermore used for diseases in which blood coagula-tion makes a crucial contribution toward the course of the disease or repre-sents a source of secondary pathology, such as, for example, in cancer, including metastasis, inflammatory diseases, including arthritis, and dia-betes.

The compounds according to the invention are furthermore used for the treatment of migraine (F.Morales-Asin et al., Headache, 40, 2000, 45-47). The invention also relates to the use of compounds of the formula I and pharmaceutically usable derivatives, solvates, salts and stereoisomers thereof, including mixtures thereof in all ratios,

for the preparation of a medicament for the prevention and treatment of thromboembolic diseases and/or thromboses as a consequence of a surgical intervention, genetically caused diseases with increased thrombosis suitability, diseases of the arterial and venous vascular system, cardiac insufficiency, atrial fibrillation, thrombophilia, tinnitus and/or sepsis. Preference is given to uses where the surgical interventions are selected from the group

thorax operations, operations in the abdominal region, orthopaedic inter-ventions, hip and knee joint replacement, CABG (coronary artery bypass grafting), artificial heart-valve replacement, operations with use of a heart-lung machine, vascular surgery, organ transplants and use of central vein catheters.

The use of anticoagulants in tinnitus therapy is described by R. Mora et al. in International Tinnitus Journal (2003), 9(2), 109-111.

The invention also relates to the use of the compounds of the formula I for the preparation of a medicament for the prevention and treatment of thromboembolic diseases and/or thromboses in adults and children.

In the treatment of the diseases described, the compounds according to the invention are also employed in combination with other thrombolytically effective compounds, such as, for example, with tissue plasminogen activator t-PA, modified t-PA, streptokinase or urokinase. The compounds according to the invention are either administered at the same time as or before or after the other said substances.

Particular preference is given to the simultaneous administration with aspirin in order to prevent recurrence of the thrombus formation.

The compounds according to the invention are also used in combination with blood platelet glycoprotein receptor (IIb/IIIa) antagonists, which inhibit blood platelet aggregation.

The invention relates to the compounds of the formula I and salts thereof and to a process for the preparation of compounds of the formula I according to claims 1-16 and pharmaceutically usable derivatives, solvates, salts and stereoisomers thereof, characterised in that a compound of the formula II 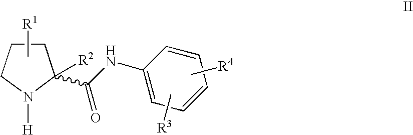
in which R¹, R², R³ and R⁴ have the meaning indicated in Claim 1,
is reacted with a compound of the formula III
Y—X—(CH₂)ₙ-L III
in which
L denotes Cl, Br, I or a free or reactively functionally modified OH group and
X, Y and n have the meanings indicated in Claim 1,
   and/or
   a base or acid of the formula I is converted into one of its salts.

The invention also relates to the optically active forms (stereoisomers), the enantiomers, the racemates, the diastereomers and the hydrates and sotvates of these compounds. The term “solvates of the compounds” is taken to mean adductions of inert solvent molecules onto the compounds which form owing to their mutual attractive force. Solvates are, for example, mono- or dihydrates or alcoholates.

The term “pharmaceutically usable derivatives” is taken to mean, for example, the salts of the compounds according to the invention and also so-called prodrug compounds.

The term “prodrug derivatives” is taken to mean compounds of the formula I which have been modified with, for example, alkyl or acyl groups, sugars or oligopeptides and which are rapidly cleaved in the organism to form the active compounds according to the invention.

These also include biodegradable polymer derivatives of the compounds according to the invention, as described, for example, in Int. J. Pharm. 115, 61-67 (1995).

The invention also relates to mixtures of the compounds of the formula I according to the invention, for example mixtures of two diastereomers, for example in the ratio 1:1, 1:2, 1.3, 1:4, 1:5, 1:10, 1:100 or 1:1000.

These are particularly preferably mixtures of stereoisomeric compounds.

For all radicals which occur more than once, such as, for example, A, their meanings are independent of one another.

Above and below, the radicals or parameters R¹, R², R³, R⁴, X, Y and n have the meanings indicated under the formula I, unless expressly indicated otherwise.

A denotes alkyl, is unbranched (linear) or branched, and has 1, 2, 3, 4, 5, 6, 7, 8, 9 or 10 C atoms. A preferably denotes methyl, furthermore ethyl, propyl, isopropyl, butyl, isobutyl, sec-butyl or tert-butyl, furthermore also pentyl, 1-, 2- or 3-methylbutyl, 1,1-, 1,2- or 2,2-dimethylpropyl, 1-ethyl-propyl, hexyl, 1-, 2-, 3- or 4-methylpentyl, 1,1-, 1,2-, 1,3-, 2,2-, 2,3- or 3,3-dimethylbutyl, 1- or 2-ethylbutyl, 1-ethyl-1-methylpropyl, 1-ethyl-2-methylpropyl, 1,1,2- or 1,2,2-trimethylpropyl, further preferably, for example, trifluoromethyl.

A very particularly preferably denotes alkyl having 1, 2, 3, 4, 5 or 6 C atoms, preferably methyl, ethyl, propyl, isopropyl, butyl, isobutyl, sec-butyl, tert-butyl, pentyl, hexyl, trifluoromethyl, pentafluoroethyl or 1,1,1-trifluoroethyl.

Cycloalkyl preferably denotes cyclopropyl, cyclobutyl, cyclopentyl, cyclohexyl or cycloheptyl.

R¹ preferably denotes H, ═O, Hal, A, OH, OA or —O—(CH₂)ₘ—OA, particularly preferably OH; OA, such as, for example, methoxy; or —O—(CH₂)ₘ—OA, such as, for example, methoxyethoxy; very particularly preferably H.

R² preferably denotes H.

R³ preferably denotes H, methyl, F or Cl, very particularly preferably H.

R⁴ preferably denotes 2-oxopiperidin-1-yl, 2-oxopyrrolidin-1-yl, 2-oxo-1H-pyridin-1-yl, 3-oxomorpholin-4-yl, 4-oxo-1H-pyridin-1-yl, 2-oxo-1H-pyrazin-1-yl, 2-oxoimidazolidin-1-yl, 2-iminopiperidin-1-yl, 2-iminopyrrolidin-1-yl, 3-iminomorpholin-4-yl, 2-iminoimidazolidin-1-yl, 2-imino-1H-pyrazin-1-yl, 2-oxopiperazin-1-yl or 3-oxo-2H-pyridazin-2-yl; particularly preferably 2-oxopiperidin-1-yl, 2-oxopyrrolidin-1-yl, 2-oxo-1H-pyridin-1-yl or 3-oxomorpholin-4-yl; very particular preference is given to 3-oxomorpholin-4-yl.

The compounds of the formula I can have one or more centres of chirality and can therefore occur in various stereoisomeric forms. The formula I covers all these forms.

Accordingly, the invention relates, in particular, to the compounds of the formula I in which at least one of the said radicals has one of the preferred meanings indicated above. Some preferred groups of compounds can be expressed by the following sub-formulae Ia to Iy, which conform to the formula I and in which the radicals not designated in greater detail have the meaning indicated under the formula I, but in which

in Ia R⁴ denotes 3-oxomorpholin-4-yl;

in Ib R¹ denotes H,

R² denotes H,
R³ denotes H,
R⁴ denotes 3-oxomorpholin-4-yl,
   and pharmaceutically usable derivatives, solvates, salts and stereoisomers thereof, including mixtures thereof in all ratios.

The compounds of the formula I and also the starting materials for the preparation thereof are, in addition, prepared by methods known per se, as described in the literature (for example in the standard works, such as Houben-Weyl, Methoden der organischen Chemie [Methods of Organic Chemistry], Georg-Thieme-Verlag, Stuttgart), to be precise under reaction conditions which are known and suitable for the said reactions. Use can also be made here of variants which are known per se, but are not mentioned here in greater detail.

If desired, the starting materials can also be formed in situ so that they are not isolated from the reaction mixture, but instead are immediately converted further into the compounds of the formula I.

The starting compounds of the formulae II and III are generally known. If they are novel, they can, however, be prepared by methods known per se.

Compounds of the formula I can preferably be obtained by reacting compounds of the formula II with compounds of the formula III.

The reaction is generally carried out in an inert solvent, in the presence of an acid-binding agent, preferably an alkali or alkaline earth metal hydroxyide, carbonate or bicarbonate or another salt of a weak acid of the alkali or alkaline earth metals, preferably of potassium, sodium, calcium or caesium. It may also be favourable to add an organic base, such as triethyl-amine, dimethylaniline, pyridine or quinoline, or an excess of the phenol component of the formula II or of the alkylation derivative of the formula III. Depending on the conditions used, the reaction time is between a few minutes and 14 days, the reaction temperature is between about 0° and 150°, normally between 20° and 130°.

Examples of suitable inert solvents are hydrocarbons, such as hexane, petroleum ether, benzene, toluene or xylene; chlorinated hydrocarbons, such as trichloroethylene, 12-dichloroethane, tetrachloromethane, chloro-form or dichloromethane; alcohols, such as methanol, ethanol, isopropanol, n-propanol, n-butanol or tert-butanol; ethers, such as diethyl ether, diisopropyl ether, tetrahydrofuran (THF) or dioxane; glycol ethers, such as ethylene glycol monomethyl or monoethyl ether, ethylene glycol dimethyl ether (diglyme); ketones, such as acetone or butanone; amides, such as acetamide, dimethylacetamide or dimethylformamide (DMF); nitriles, such as acetonitrile; sulfoxides, such as dimethyl sulfoxide (DMSO); carbon di-sulfide; carboxylic acids, such as formic acid or acetic acid; nitro compounds, such as nitromethane or nitrobenzene; esters, such as ethyl ace-tate, or mixtures of the said solvents.

In the compounds of the formula III, L preferably denotes Cl, Br, I or a free or reactively modified OH group, such as, for example, an activated ester, an imidazolide or alkylsulfonyloxy having 1-6 C atoms (preferably methyl-sulfonyloxy or trifluoromethylsulfonyloxy) or arylsulfonyloxy having 6-10 C atoms (preferably phenyl- or p-tolylsulfonyloxy).

Radicals of this type for activation of the carboxyl group in typical acylation reactions are described in the literature (for example in the standard works, such as Houben-Weyl, Methoden der organischen Chemie [Methods of Organic Chemistry], Georg-Thieme-Verlag, Stuttgart;).

Activated esters are advantageously formed in situ, for example through addition of HOBt or N-hydroxysuccinimide.

Pharmaceutical Salts and Other Forms

The said compounds of the formula I can be used in their final non-salt form. On the other hand, the present invention also relates to the use of these compounds in the form of their pharmaceutically acceptable salts, which can be derived from various organic and inorganic acids and bases by procedures known in the art. Pharmaceutically acceptable salt forms of the compounds of the formula I are for the most part prepared by conventional methods. If the compound of the formula I contains a carboxyl group, one of its suitable salts can be formed by reacting the compound with a suitable base to give the corresponding base-addition salt. Such bases are, for example, alkali metal hydroxides, including potassium hydroxide, sodium hydroxide and lithium hydroxide; alkaline earth metal hydroxides, such as barium hydroxide and calcium hydroxide; alkali metal alkoxides, for example potassium ethoxide and sodium propoxide; and various organic bases, such as piperidine, diethanolamine and N-methyl-glutamine. The aluminium salts of the compounds of the formula I are likewise included. In the case of certain compounds of the formula I, acid-addition salts can be formed by treating these compounds with pharmaceutically acceptable organic and inorganic acids, for example hydrogen halides, such as hydrogen chloride, hydrogen bromide or hydrogen iodide, other mineral acids and corresponding salts thereof, such as sulfate, nitrate or phosphate and the like, and alkyl- and monoaryl-sulfonates, such as ethanesulfonate, toluenesulfonate and benzene-sulfonate, and other organic acids and corresponding salts thereof such as acetate, trifluoroacetate, tartrate, maleate, succinate, citrate, benzoate, salicylate, ascorbate and the like. Accordingly, pharmaceutically acceptable acid-addition salts of the compounds of the formula I include the following: acetate, adipate, alginate, arginate, aspartate, benzoate, benzenesulfonate (besylate), bisulfate, bisulfite, bromide, butyrate, camphorate, camphorsulfonate, caprylate, chloride, chlorobenzoate, citrate, cyclopentanepropionate, digluconate, dihydrogenphosphate, dinitrobenzoate, dodecylsulfate, ethanesulfonate, fumarate, galacterate (from mucic acid), galacturonate, glucoheptanoate, gluconate, glutamate, glycerophosphate, hemisuccinate, hemisulfate, heptanoate, hexanoate, hippurate, hydrochloride, hydrobromide, hydroiodide, 2-hydroxyethane-sulfonate, iodide, isethionate, isobutyrate, lactate, lactobionate, malate, maleate, malonate, mandelate, metaphosphate, methanesulfonate, methylbenzoate, monohydrogenphosphate, 2-naphthalenesulfonate, nicotinate, nitrate, oxalate, oleate, palmoate, pectinate, persulfate, phenylacetate, 3-phenylpropionate, phosphate, phosphonate, phthalate, but this does not represent a restriction.

Furthermore, the base salts of the compounds of the formula I include aluminium, ammonium, calcium, copper, iron(III), iron(II), lithium, magnesium, manganese(III), manganese(II), potassium, sodium and zinc salts, but this is not intended to represent a restriction. Of the abovementioned salts, preference is given to ammonium; the alkali metal salts sodium and potassium, and the alkaline earth metal salts calcium and magnesium. Salts of the compounds of the formula I which are derived from pharmaceutically acceptable organic non-toxic bases include salts of primary, secondary and tertiary amines, substituted amines, also including naturally occurring substituted amines, cyclic amines, and basic ion exchanger res-ins, for example arginine, betaine, caffeine, chloroprocaine, choline, N,N′-dibenzylethylenediamine (benzathine), dicyclohexylamine, diethanolamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lido-caine, lysine, meglumine, N-methyl-D-glucamine, morpholine, piperazine, piperidine, polyamine resins, procaine, purines, theobromine, triethanol-amine, triethylamine, trimethylamine, tripropylamine and tris(hydroxy-methyl)methylamine (tromethamine), but this is not intended to represent a restriction.

Compounds of the formula I of the present invention which contain basic nitrogen-containing groups can be quaternised using agents such as (C₁-C₄)alkyl halides, for example methyl, ethyl, isopropyl and tert-butyl chloride, bromide and iodide; di(C₁-C₄)alkyl sulfates, for example dimethyl, diethyl and diamyl sulfate; (C₁₀-C₁₈)alkyl halides, for example decyl, dode-cyl, lauryl, myristyl and stearyl chloride, bromide and iodide; and aryl-(C₁-C₄)alkyl halides, for example benzyl chloride and phenethyl bromide. Both water- and oil-soluble compounds of the formula I can be prepared using such salts.

The above-mentioned pharmaceutical salts which are preferred include acetate, trifluoroacetate, besylate, citrate, fumarate, gluconate, hemisuccinate, hippurate, hydrochloride, hydrobromide, isethionate, mandelate, me-glumine, nitrate, oleate, phosphonate, pivalate, sodium phosphate, stea-rate, sulfate, sulfosalicylate, tartrate, thiomalate, tosylate and trometh-amine, but this is not intended to represent a restriction.

The acid-addition salts of basic compounds of the formula I are prepared by bringing the free base form into contact with a sufficient amount of the desired acid, causing the formation of the salt in a conventional manner. The free base can be regenerated by bringing the salt form into contact with a base and isolating the free base in a conventional manner. The free base forms differ in a certain respect from the corresponding salt forms thereof with respect to certain physical properties, such as solubility in polar solvents; for the purposes of the invention, however, the salts otherwise correspond to the respective free base forms thereof.

As mentioned, the pharmaceutically acceptable base-addition salts of the compounds of the formula I are formed with metals or amines, such as alkali metals and alkaline earth metals or organic amines. Preferred metals are sodium, potassium, magnesium and calcium. Preferred organic amines are N,N′-dibenzylethylenediamine, chloroprocaine, choline, diethanolamine, ethylenediamine, 1N-methyl-D-glucamine and procaine.

The base-addition salts of acidic compounds of the formula I are prepared by bringing the free acid form into contact with a sufficient amount of the desired base, causing the formation of the salt in a conventional manner. The free acid can be regenerated by bringing the salt form into contact with an acid and isolating the free acid in a conventional manner. The free acid forms differ in a certain respect from the corresponding salt forms thereof with respect to certain physical properties, such as solubility in polar solvents; for the purposes of the invention, however, the salts otherwise correspond to the respective free acid forms thereof.

If a compound of the formula I contains more than one group which is capable of forming pharmaceutically acceptable salts of this type, the formula also encompasses multiple salts. Typical multiple salt forms include, for example, bitartrate, diacetate, difumarate, dimeglumine, diphosphate, disodium and trihydrochloride, but this is not intended to represent a restriction.

With regard to that stated above, it can be seen that the term “pharmaceutically acceptable salt” in the present connection is taken to mean an active ingredient which comprises a compound of the formula I in the form of one of its salts, in particular if this salt form imparts improved pharmacokinetic properties on the active ingredient compared with the free form of the active ingredient or any other salt form of the active ingredient used earlier. The pharmaceutically acceptable salt form of the active ingredient can also provide this active ingredient for the first time with a desired pharmacokinetic property which it did not have earlier and can even have a positive influence on the pharmacodynamics of this active ingredient with respect to its therapeutic efficacy in the body.

Owing to their molecular structure, compounds of the formula I according to the invention can be chiral and can accordingly occur in various enantiomeric forms. They can therefore exist in racemic or in optically active form.

Since the pharmaceutical activity of the racemates or stereoisomers of the compounds according to the invention may differ, it may be desirable to use the enantiomers. In these cases, the end product or even the interme-diates can be separated into enantiomeric compounds by chemical or physical measures known to the person skilled in the art or even employed as such in the synthesis.

In the case of racemic amines, diastereomers are formed from the mixture by reaction with an optically active resolving agent. Examples of suitable resolving agents are optically active acids, such as the R and S forms of tartaric acid, diacetyltartaric acid, dibenzoyltartaric acid, mandelic acid, malic acid, lactic acid, suitably N-protected amino acids (for example N-benzoylproline or N-benzenesulfonylproline), or the various optically active camphorsulfonic acids. Also advantageous is chromatographic en-antiomer resolution with the aid of an optically active resolving agent (for example dinitrobenzoylphenylglycine, cellulose triacetate or other derivatives of carbohydrates or chirally derivatised methacrylate polymers immobilised on silica gel). Suitable eluents for this purpose are aqueous or al-coholic solvent mixtures, such as, for example, hexane/isopropanolf acetonitrile, for example in the ratio 82:15:3.

The invention furthermore relates to the use of the compounds of the formula I and/or physiologically acceptable salts thereof for the preparation of a medicament (pharmaceutical composition), in particular by non-chemical methods. In this case, they can be converted into a suitable dosage form together with at least one solid, liquid and/or semi-liquid excipient or adjuvant and optionally in combination with one or more further active ingredients.

The invention furthermore relates to medicaments comprising at least one compound of the formula I and/or pharmaceutically usable derivatives, solvates, salts and stereoisomers thereof, including mixtures thereof in all ratios, and optionally excipients and/or adjuvants.

These compositions can be used as medicaments in human or veterinary medicine.

Pharmaceutical formulations can be administered in the form of dosage units which comprise a predetermined amount of active ingredient per dosage unit. Such a unit can comprise, for example, 0.5 mg to 1 g, preferably 1 mg to 700 mg, particularly preferably 5 mg to 100 mg, of a compound according to the invention, depending on the disease condition treated, the method of administration and the age, weight and condition of the patient, or pharmaceutical formulations can be administered in the form of dosage units which comprise a predetermined amount of active ingredient per dosage unit. Preferred dosage unit formulations are those which comprise a daily dose or part-dose, as indicated above, or a corresponding fraction thereof of an active ingredient. Furthermore, pharmaceutical formulations of this type can be prepared using a process which is generally known in the pharmaceutical art.

Pharmaceutical formulations can be adapted for administration via any desired suitable method, for example by oral (including buccal or sublingual), rectal, nasal, topical (including buccal, sublingual or transdermal), vaginal or parenteral (including subcutaneous, intramuscular, intravenous or intra-dermal) methods. Such formulations can be prepared using all processes known in the pharmaceutical art by, for example, combining the active ingredient with the excipient(s) or adjuvant(s).

Pharmaceutical formulations adapted for oral administration can be administered as separate units, such as, for example, capsules or tablets; powders or granules; solutions or suspensions in aqueous or non-aqueous liquids; edible foams or foam foods; or oil-in-water liquid emulsions or water-in-oil liquid emulsions.

Thus, for example, in the case of oral administration in the form of a tablet or capsule, the active-ingredient component can be combined with an oral, non-toxic and pharmaceutically acceptable inert excipient, such as, for example, ethanol, glycerol, water and the like. Powders are prepared by comminuting the compound to a suitable fine size and mixing it with a pharmaceutical excipient comminuted in a similar manner, such as, for ex-ample, an edible carbohydrate, such as, for example, starch or mannitol. A flavour, preservative, dispersant and dye may likewise be present.

Capsules are produced by preparing a powder mixture as described above and filling shaped gelatine shells therewith. Glidants and lubricants, such as, for example, highly disperse silicic acid, talc, magnesium stearate, cal-cium stearate or polyethylene glycol in solid form, can be added to the powder mixture before the filling operation. A disintegrant or solubiliser, such as, for example, agar-agar, calcium carbonate or sodium carbonate, may likewise be added in order to improve the availability of the medicament after the capsule has been taken.

In addition, if desired or necessary, suitable binders, lubricants and disintegrants as well as dyes can likewise be incorporated into the mixture. Suitable binders include starch, gelatine, natural sugars, such as, for example, glucose or beta-lactose, sweeteners made from maize, natural and synthetic rubber, such as, for example, acacia, tragacanth or sodium alginate, carboxymethylcellulose, polyethylene glycol, waxes, and the like. The lubricants used in these dosage forms include sodium oleate, sodium stearate, magnesium stearate, sodium benzoate, sodium acetate, sodium chloride and the like. The disintegrants include, without being restricted thereto, starch, methylcellulose, agar, bentonite, xanthan gum and the like. The tablets are formulated by, for example, preparing a powder mixture, granulating or dry-pressing the mixture, adding a lubricant and a disintegrant and pressing the entire mixture to give tablets. A powder mixture is prepared by mixing the compound comminuted in a suitable manner with a diluent or a base, as described above, and optionally with a binder, such as, for example, carboxymethylcellulose, an alginate, gelatine or polyvinyl-pyrrolidone, a dissolution retardant, such as, for example, paraffin, an absorption accelerator, such as, for example, a quaternary salt, and/or an absorbant, such as, for example, bentonite, kaolin or dicalcium phosphate.

The powder mixture can be granulated by wetting it with a binder, such as, for example, syrup, starch paste, acadia mucilage or solutions of cellulose or polymer materials and pressing it through a sieve. As an alternative to granulation, the powder mixture can be run through a tableting machine, giving lumps of non-uniform shape which are broken up to form granules. The granules can be lubricated by addition of stearic acid, a stearate salt, talc or mineral oil in order to prevent sticking to the tablet casting moulds. The lubricated mixture is then pressed to give tablets. The active ingredients can also be combined with a free-flowing inert excipient and then pressed directly to give tablets without carrying out the granulation or dry-pressing steps. A transparent or opaque protective layer consisting of a shellac sealing layer, a layer of sugar or polymer material and a gloss layer of wax may be present. Dyes can be added to these coatings in order to be able to differentiate between different dosage units.

Oral liquids, such as, for example, solution, syrups and elixirs, can be prepared in the form of dosage units so that a given quantity comprises a pre-specified amount of the compounds. Syrups can be prepared by dissolving the compounds in an aqueous solution with a suitable flavour, while elixirs are prepared using a non-toxic alcoholic vehicle. Suspensions can be formulated by dispersion of the compounds in a non-toxic vehicle. Solubilisers and emulsifiers, such as, for example, ethoxylated isostearyl alcohols and polyoxyethylene sorbitol ethers, preservatives, flavour additives, such as, for example, peppermint oil or natural sweeteners or saccharin, or other artificial sweeteners and the like, can likewise be added.

The dosage unit formulations for oral administration can, if desired, be encapsulated in microcapsules. The formulation can also be prepared in such a way that the release is extended or retarded, such as, for example, by coating or embedding of particulate material in polymers, wax and the like.

The compounds of the formula I and salts, solvates and physiologically functional derivatives thereof and the other active ingredients can also be administered in the form of liposome delivery systems, such as, for example, small unilamellar vesicles, large unilamellar vesicles and multilamellar vesicles. Liposomes can be formed from various phospholipids, such as, for example, cholesterol, stearylamine or phosphatidylcholines.

The compounds of the formula I and the salts, solvates and physiologically functional derivatives thereof and the other active ingredients can also be delivered using monoclonal antibodies as individual carriers to which the compound molecules are coupled. The compounds can also be coupled to soluble polymers as targeted medicament carriers. Such polymers may encompass polyvinylpyrrolidone, pyran copolymer, polyhydroxypropyl-methacrylamidophenol, polyhydroxyethylaspartamidophenol or polyethyl-ene oxide polylysine, substituted by palmitoyl radicals. The compounds may furthermore be coupled to a class of biodegradable polymers which are suitable for achieving controlled release of a medicament, for example polylactic acid, poly-epsilon-caprolactone, polyhydroxybutyric acid, poly-orthoesters, polyacetals, polydihydroxypyrans, polycyanoacrylates and crosslinked or amphipathic block copolymers of hydrogels.

Pharmaceutical formulations adapted for transdermal administration can be administered as independent plasters for extended, close contact with the epidermis of the recipient. Thus, for example, the active ingredient can be delivered from the plaster by iontophoresis, as described in general terms in Pharmaceutical Research, 3(6), 318 (1986).

Pharmaceutical compounds adapted for topical administration can be formulated as ointments, creams, suspensions, lotions, powders, solutions, pastes, gels, sprays, aerosols or oils.

For the treatment of the eye or other external tissue, for example mouth and skin, the formulations are preferably applied as topical ointment or cream. In the case of formulation to give an ointment, the active ingredient can be employed either with a paraffinic or a water-miscible cream base. Alternatively, the active ingredient can be formulated to give a cream with an oil-in-water cream base or a water-in-oil base.

Pharmaceutical formulations adapted for topical application to the eye include eye drops, in which the active ingredient is dissolved or suspended in a suitable carrier, in particular an aqueous solvent.

Pharmaceutical formulations adapted for topical application in the mouth encompass lozenges, pastilles and mouthwashes.

Pharmaceutical formulations adapted for rectal administration can be administered in the form of suppositories or enemas.

Pharmaceutical formulations adapted for nasal administration in which the carrier substance is a solid comprise a coarse powder having a particle size, for example, in the range 20-500 microns, which is administered in the manner in which snuff is taken, i.e. by rapid inhalation via the nasal passages from a container containing the powder held close to the nose. Suitable formulations for administration as nasal spray or nose drops with a liquid as carrier substance encompass active-ingredient solutions in water or oil.

Pharmaceutical formulations adapted for administration by inhalation encompass finely particulate dusts or mists, which can be generated by various types of pressurised dispensers with aerosols, nebulisers or insufflators.

Pharmaceutical formulations adapted for vaginal administration can be administered as pessaries, tampons, creams, gels, pastes, foams or spray formulations.

Pharmaceutical formulations adapted for parenteral administration include aqueous and non-aqueous sterile injection solutions comprising antioxidants, buffers, bacteriostatics and solutes, by means of which the formulation is rendered isotonic with the blood of the recipient to be treated; and aqueous and non-aqueous sterile suspensions, which may comprise suspension media and thickeners. The formulations can be administered in single-dose or multidose containers, for example sealed ampoules and vials, and stored in the freeze-dried (lyophilised) state, so that only the addition of the sterile carrier liquid, for example water for injection purposes, immediately before use is necessary.

Injection solutions and suspensions prepared in accordance with the recipe can be prepared from sterile powders, granules and tablets.

It goes without saying that, in addition to the above particularly mentioned constituents, the formulations may also comprise other agents usual in the art with respect to the particular type of formulation; thus, for example, formulations which are suitable for oral administration may comprise flavours.

A therapeutically effective amount of a compound of the formula I and of the other active ingredient depends on a number of factors, including, for example, the age and weight of the animal, the precise disease condition which requires treatment, and its severity, the nature of the formulation and the method of administration, and is ultimately determined by the treating doctor or vet. However, an effective amount of a compound is generally in the range from 0.1 to 100 mg/kg of body weight of the recipient (mammal) per day and particularly typically in the range from 1 to 10 mg/kg of body weight per day. Thus, the actual amount per day for an adult mammal weighing 70 kg is usually between 70 and 700 mg, where this amount can be administered as an individual dose per day or usually in a series of part-doses (such as, for example, two, three, four, five or six) per day, so that the total daily dose is the same. An effective amount of a salt or solvate or of a physiologically functional derivative thereof can be determined as the fraction of the effective amount of the compound per se.

The compounds of the formula I and physiologically acceptable salts thereof can be used in the combating and prevention of thromboembolic diseases, such as thrombosis, myocardial infarction, arteriosclerosis, inflammation, apoplexy, angina pectoris, restenosis after angioplasty, claudicatio intermittens, migraine, tumours, tumour diseases and/or tumour metastases.

The invention furthermore relates to the use of compounds according to one or more of Claims 1-27, in combination with at least one further medicament active ingredient.

The further medicament active ingredients are preferably selected from the group of the antithrombotics, antiarrhythmics, contraceptives, phospho-diesterase V inhibitors.

The antithrombotic is preferably selected from the group of the vitamin K antagonists, heparin compounds, thrombocyte aggregation inhibitors, enzymes, other antithrombotic agents, blood platelet glycoprotein receptor (IIb/IIIa) antagonists, thromboxane antagonists, thrombocyte adhesion inhibitors.

The vitamin K antagonists are preferably selected from the group dicoumarol, phenindione, warfarin, phenprocoumon, acenocoumarol, ethyl bis-coumacetate, clorindione, diphenadione, tioclomarol.

The heparin compounds are preferably selected from the group heparin, antithrombin III, dalteparin, enoxaparin, nadroparin, parnaparin, reviparin, danaparoid, tinzaparin, sulodexide.

The thrombocyte aggregation inhibitors are preferably selected from the group ditazole, cloricromen, picotamide, clopidogrel, ticlopidine, acetylsalicylic acid, dipyridamole, calcium carbassalate, epoprostenol, indo-bufen, iloprost, abciximab, tirofiban, aloxiprin, intrifiban.

The enzymes are preferably selected from the group streptokinase, alteplase, anistreplase, urokinase, fibrinolysin, brinase, reteplase, saruplase.

The other antithrombotic agents are preferably selected from the group defibrotide, desirudin, lepirudin.

The thromboxane antagonists are preferably selected from the group ramatroban, equalen sodium, seratrodast.

The antiarrhythmics are preferably selected from the group

a) quinidine, disopyramide, ajmaline, detajmium,

b) lidocaine, mexiletine, phenyloin, tocamide,

c) propafenone, flecainide,

d) metoprolol, esmolol, propranolol, atenolol, oxprenolol,

e) amiodarone, sotalol,

f) diltiazem, verapamil, gallopamil,

g) adenosine, orciprenaline, ipratropium,

h) cardiac glycosides.

The contraceptives are preferably selected from the group desogestrel, medroxyprogesterone acetate, levonorgestrel, etonogestrel, norethisterone enantate.

The PDE V inhibitors are preferably selected are from the group

a) sildenafil (Viagra®), tadalafil (Clalis®), vardenafil (Levitra®),

b) the compounds of the formula I described in WO 99/55708 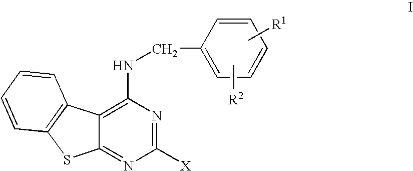
in which
R¹, R² each, independently of one another, denote H, A, OA, OH or Hal,
R¹ and R² together also denote alkylene having 3-5 C atoms, —O—CH₂—CH₂—, —CH₂—O—CH₂—, —O—CH₂—O— or —O—CH₂—CH₂—O—,
X denotes mono-R⁷-substituted R⁴, R⁵ or R⁶,
R⁴ denotes linear or branched alkylene having 1-10 C atoms, in which one or two CH₂ groups may be replaced by —CH═CH— groups,
R⁵ denotes cycloalkyl or cycloalkylalkylene having 5-12 C atoms,
R⁶ denotes phenyl or phenylmethyl,
R⁷ denotes COOH, COOA, CONH₂, CONHA, CON(A)₂ or CN,
A denotes alkyl having 1 to 6 C atoms and
Hal denotes F, Cl, Br or I,
   and/or physiologically acceptable salts and/or solvates thereof,
   c) the compounds of the formula I described in WO 99/28325 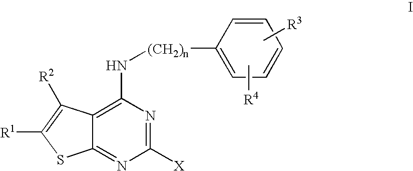
   in which
R¹, R² each, independently of one another, denote H, A or Hal, where one of the radicals R¹ or R² is always ≠H,
R¹ and R² together also denote alkylene having 3-5 C atoms,
R³, R⁴ each, independently of one another, denote Hr A, OH, OA or Hal,
R³ and R⁴ together also denote alkylene having 3-5 C atoms, —O—CH₂—CH₂—, —O—CH₂—O— or —O—CH₂—CH₂—O—,
X denotes mono-R⁷-substituted R⁵ or R⁶,
R⁵ denotes linear or branched alkylene having 1-10 C atoms, in which one or two CH₂ groups may be replaced by —CH═CH— groups, or
   —C₆H₄—(CH₂)ₘ—,
R⁶ denotes cycloalkylalkylene having 6-12 C atoms,
R⁷ denotes COOH, COOA, CONH₂, CONHA, CON(A)₂ or CN,
A denotes alkyl having 1 to 6 C atoms,
Hal denotes F, Cl, Br or I,
m denotes 1 or 2 and
n denotes 0, 1, 2 or 3,
   and/or physiologically acceptable salts and/or solvates thereof.

Preferred antithrombotics are furthermore the blood platelet glycoprotein receptor (IIb/IIIa) antagonists, which inhibit blood platelet aggregation. Preferred compounds are described, for example, in EP 0 623 615 B1 on page 2 or in EP 0 741 133 A2, page 2, line 2, to page 4, line 56.

A further medicament active ingredient is preferably also aspirin.

The invention also relates to a set (kit) consisting of separate packs of
(a) an effective amount of a compound of the formula I and/or pharmaceutically usable derivatives, solvates, salts and stereoisomers thereof, including mixtures thereof in all ratios, and
(b) an effective amount of a further medicament active ingredient.

The set comprises suitable containers, such as boxes or cartons, individual bottles, bags or ampoules. The set may, for example, comprise separate ampoules, each containing an effective amount of a compound of the formula I and/or pharmaceutically usable derivatives, solvates and stereoisomers thereof, including mixtures thereof in all ratios, and an effective amount of a further medicament active ingredient in dissolved or lyophilised form.

The invention furthermore relates to the use of compounds of the formula I and/or pharmaceutically usable derivatives, solvates, salts and stereoisomers thereof, including mixtures thereof in all ratios, for the preparation Ski of a medicament for the treatment of thromboses, myocardial infarction, arteriosclerosis, inflammation, apoplexy, angina pectoris, restenosis after angioplasty, claudicatio intermittens, migraine, tumours, tumour diseases and/or tumour metastases,

for the prevention and treatment of thromboembolic diseases and/or thromboses as a consequence of a surgical intervention, genetically caused diseases having increased thrombosis suitability, diseases of the arterial and venous vascular system, cardiac insufficiency, atrial fibrillation, thrombophilia, tinnitus and/or sepsis,

in combination with at least one further medicament active ingredient.

The invention furthermore relates to a medicament comprising a compound of the formula I and/or pharmaceutically usable derivatives, solvates, salts and stereoisomers thereof, including mixtures thereof in all ratios, and aspirin.

The invention furthermore relates to the use of a compound of the formula I and/or pharmaceutically usable derivatives, solvates, salts and stereoisomers thereof, including mixtures thereof in all ratios,

for the preparation of a medicament for the treatment of thromboses, myocardial infarction, arteriosclerosis, inflammation, apoplexy, angina pectoris, restenosis after angioplasty, claudicatio intermittens, migraine, tumours, tumour diseases and/or tumour metastases,

for the prevention and treatment of thromboembolic diseases and/or thromboses as a consequence of a surgical intervention, genetically caused diseases having increased thrombosis suitability, diseases of the arterial and venous vascular system, cardiac insufficiency, atrial fibrillation, thrombophilia, tinnitus and/or sepsis,

in combination with aspirin.

Above and below, all temperatures are indicated in ° C. In the following examples, “conventional work-up” means: water is added if necessary, the pH is adjusted, if necessary, to values between 2 and 10, depending on the constitution of the end product, the mixture is extracted with ethyl acetate or dichloromethane, the phases are separated, the organic phase is dried over sodium sulfate and evaporated, and the product is purified by chromatography on silica gel and/or by crystallisation. Rf values on silica gel; eluent: ethyl acetatelmethanol 9:1.

Mass spectrometry (MS): EI (electron impact ionisation) M⁺

FAB (fast atom bombardment) (M+H)⁺

ESI (electrospray ionisation) (M+H)⁺ (unless indicated otherwise)

### EXAMPLE 1

### Preparation of 1-N-[(5-chlorothiophen-2-yl)aminocarbonylmethyl]-2-N-[4-(3-oxomorpholin-4-yl)phenyl]-(R)-pyrrolidine-2-carboxamide (“A1”)

1.1 0.8 (5.2 mmol) of 1-hydroxybenzotriazole hydrate, 1.12 g (5.2 mmol) of D-Boc-proline, 2 g (10.4 mmol) of N-(3-dimethylamino-propyl)-N′-ethylcarbodiimide hydrochloride (DAPECI) and 1.26 ml of N-methylmorpholine are added successively to a solution of 1.0 g (5.2 mmol) of 4-(4-aminophenyl)morpholin-3-one in 25 ml of dimethyl-formamide, and the resultant solution is stirred for 12 hours at room temperature. The reaction solution is subsequently evaporated to dry-ness in vacuo, the residue is taken up in 10 ml of 5% sodium hydrogencarbonate solution, and the sodium hydrogencarbonate solution is extracted twice with 10 ml of ethyl acetate each time. After the combined organic phases have been dried over sodium sulfate and the solvent has been stripped off, the solid residue is triturated with 20 ml of diethyl ether, giving 1.4 g of tert-butyl 2-[4-(3-oxomorpholin-4-yl)phenyl-carbamoyl]pyrrolidine-1-carboxylate as white powder; ESI 390.

1.2 40 ml of 4N hydrochloric acid in dioxane are added to a solution of 1.4 g (3.60 mmol) of tert-butyl 2-[4-(3-oxomorpholin-4-yl)phenyl-carbamoyl]pyrrolidine-1-carboxylate in 20 ml of dioxane, and the mixture is stirred for 12 hours at room temperature. The deposited precipitate is subsequently filtered off with suction and washed successively with 10 ml of each of dioxane and diethyl ether and dried in vacuo, giving 1.1 g of N-[4-(3-oxomorpholin-4-yl)phenyl]pyrrolidine-2-carboxamide hydrochloride as white powder; ESI 290.

1.3 0.61 mmol of 2-chloro-N-(5-chlorothiophen-2-yl)acetamide is added to a solution of 200 mg (0.61 mmol) of N-[4-(3-oxomorpholin-4-yl)-phenyl]pyrrolidine-2-carboxamide hydrochloride and 1 ml of triethylamine in 5 ml of methylene chloride, and the reaction solution is stirred for two hours at room temperature. The reaction solution is subsequently washed with 5 ml of each of 1N hydrochloric acid and water, and the methylene chloride solution is dried over sodium sulfate. After the solvent has been stripped off in vacuo, the crude product is recrystallised from ethanol/diethyl ether, giving 120 mg of the title compound (“A1”).

The following compounds are obtained analogously
1-N-[(5-chlorothiophen-2-yl)aminocarbonylmethyl]-2-N-[4-(3-oxomorpholin-4-yl)phenyl]-(S)-pyrrolidine-2-carboxamide,
1-N-[(4-chlorophenyl)aminocarbonylmethyl]-2-N-[4-(3-oxomorpholin-4-yl)phenyl]-(R)-pyrrolidine-2-carboxamide,
1-N-[(3-chloropyridin-6-yl)aminocarbonylmethyl]-2-N-[4-(3-oxomor-pholin-4-yl)phenyl]-(R)-pyrrolidine-2-carboxamide,
1-N-[2-(4-chlorophenyl)ethyl]-2-N-[4-(3-oxomorpholin-4-yl)phenyl]-(R)-pyrrolidine-2-carboxamide,
1-N-[(6-chlorobenzo[b]thiophen-2-yl)methyl]-2-N-[4-(3-oxomorpho-lin-4-yl)phenyl]-(R)-pyrrolidine-2-carboxamide,
1-N-[(3-(4-chlorophenyl)isoxazol-5-yl)methyl]-2-N-[4-(3-oxomorpho-lin-4-yl)phenyl]-(R)-pyrrolidine-2-carboxamide,
1-N-[(5-(4-chlorophenyl)isoxazol-3-yl)methyl]-2-N-[4-(3-oxomorpho-lin-4-yl)phenyl]-(R)-pyrrolidine-2-carboxamide,
1-N-[(3-(4-chlorophenyl)-1,2,4-oxdiazol-5-yl)methyl]-2-N-[4-(3-oxo-morpholin-4-yl)phenyl]-(R)-pyrrolidine-2-carboxamide,
1-N-[(2-(4-chlorophenyl)thiazol-5-yl)methyl]-2-N-[4-(3-oxomorpho-lin-4-yl)phenyl]-(R)-pyrrolidine-2-carboxamide,
1-N-[(3-(2-chlorothiophen-5-yl)isoxazol-5-yl)methyl]-2-N-[4-(3-oxomor-pholin-4-yl)phenyl]-(R)-pyrrolidine-2-carboxamide,
1-N-[(5-(2-chlorothiophen-5-yl)isoxazol-3-yl)methyl]-2-N-[4-(3-oxomor-pholin-4-yl)phenyl]-(R)-pyrrolidine-2-carboxamide,
1-N-[(2-(2-chlorothiophen-5-yl)-1,3,4-thiadiazol-5-yl)methyl]-2-N-[4-(3-oxomorpholin-4-yl)phenyl]-(R)-pyrrolidine-2-carboxamide,
1-N-[(2-(2-chlorothiophen-5-yl)thiazol-5-yl)methyl]-2-N-[4-(3-oxomor-pholin-4-yl)phenyl]-(R)-pyrrolidine-2-carboxamide.
   14. Examples of the Preparation of Intermediate Compounds

14.1 All compounds of the following formula VI (where R═H or methyl; n=3, 4 or 5) can be synthesised in accordance with the following scheme.

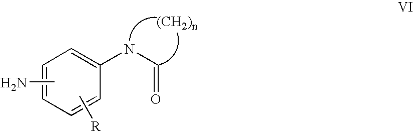

For example, synthesis of 1-(4-amino-2-methylphenyl)piperidin-2-one:

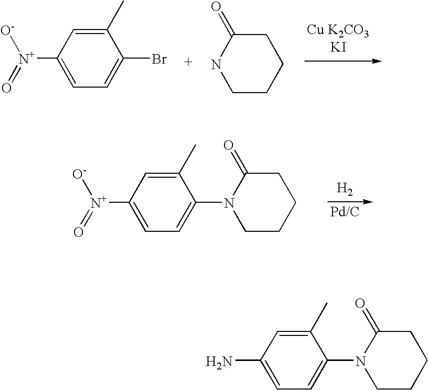

### 14.2 Synthesis of the phenylpiperidone Unit Without a Methyl Group

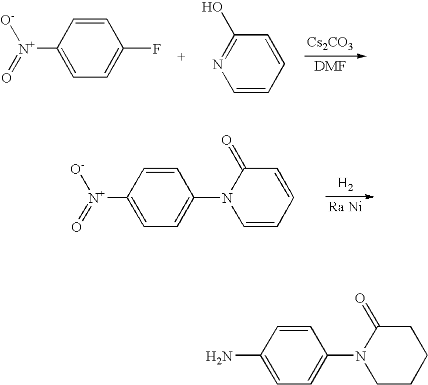

### 1-(4-Amino-2-methylphenyl)piperidin-2-one is Prepared, for Example, as Indicated Below

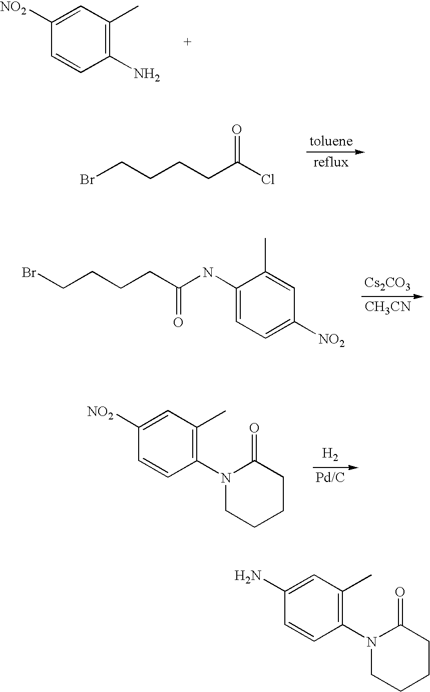

### 14.3 1-(4-Aminophenyl)-1H-pyrazin-2-one

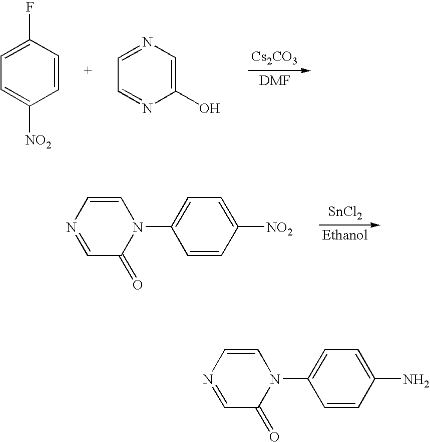

### 14.4 1-(4-Amino-2,5-dimethylphenyl)piperidin-2-one

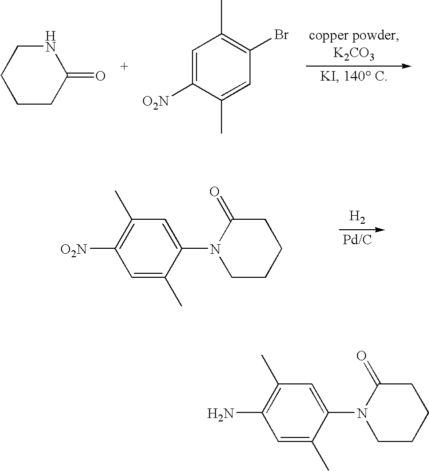

### 14.5 1-(4-Amino-3-methylphenyl)piperidin-2-one

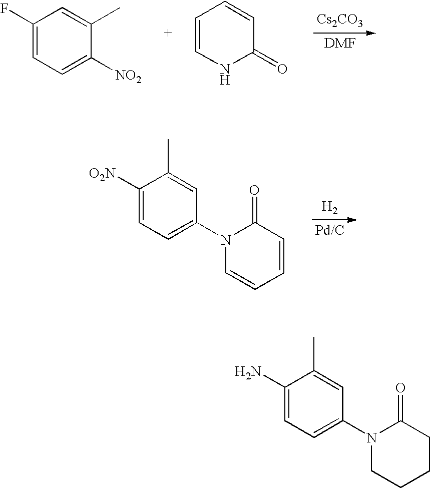

### 14.6 1-(5-Aminopyridin-2-yl)piperidin-2-one

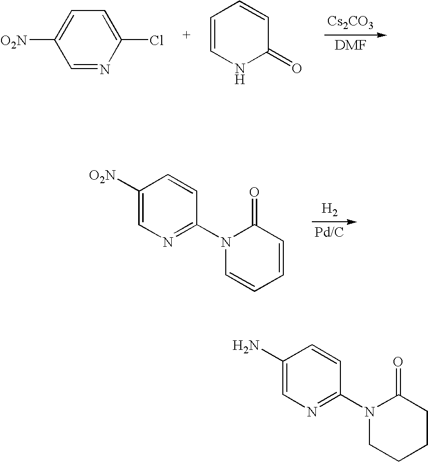

### 14.7 1-(4-Aminomethylphenyl)piperidin-2-one

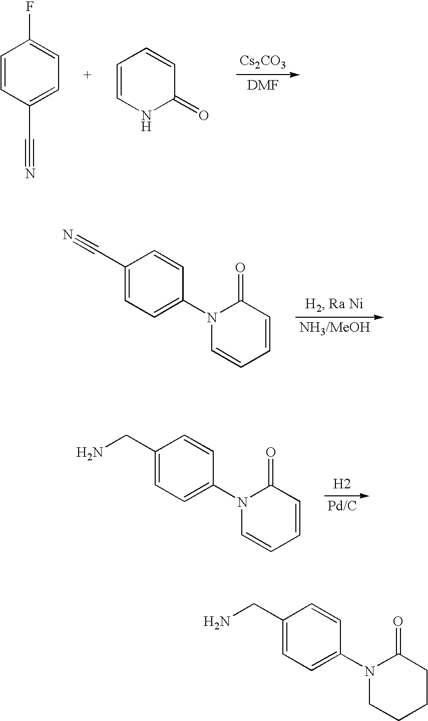

### 14.8 2-(4-Aminophenyl)-2-azabicyclo[2.2.2]octan-3-one

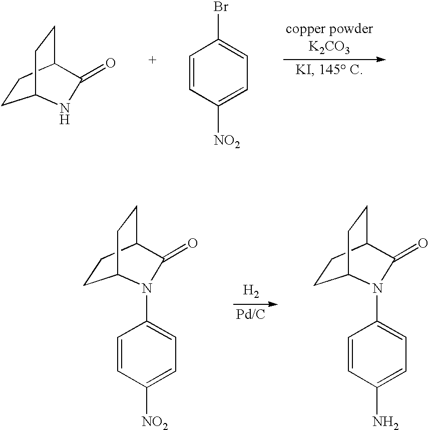

### 14.9 1-(3-Amino-6-ethylphenyl)pyrrolidin-2-one

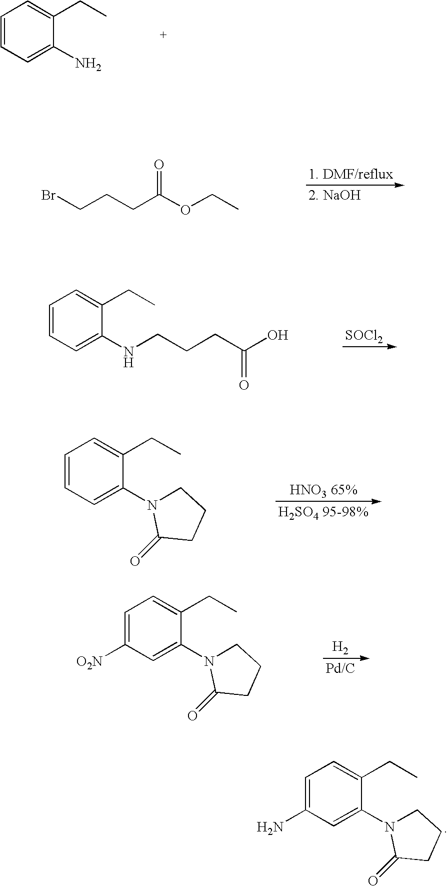

### 14.10 2-(4-Amino-2-trifluoromethylphenyl)-2-azabicyclo[2.2.2]octan-3-one

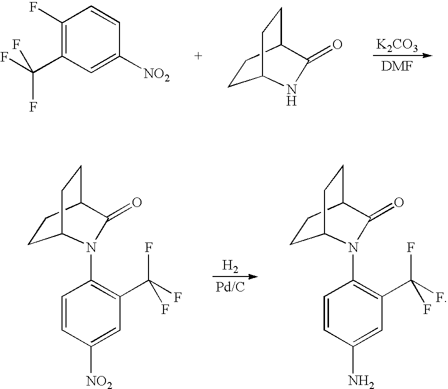

### 14.11 1-(4-Amino-3-chlorophenyl)pyrrolidin-2-one

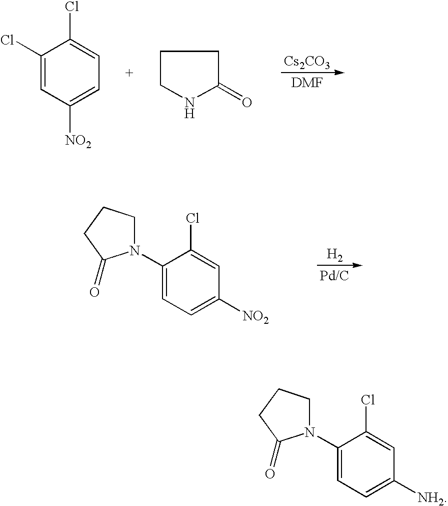

### 14.12 1-(4-Amino-2-trifluoromethylphenyl)piperidin-2-one

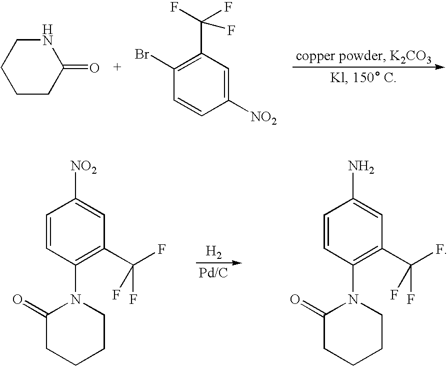

### 14.13 3-(4-Amino-2-methylphenyl)-1,3-oxazinan-2-one

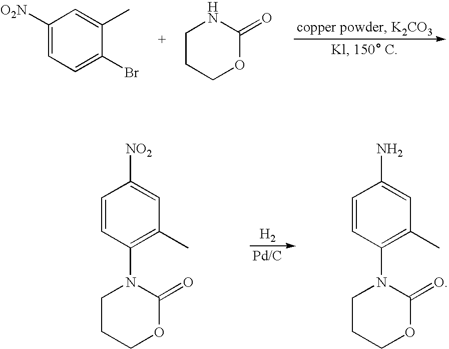

### 14.14 4-(4-Aminophenyl)morpholin-3-one

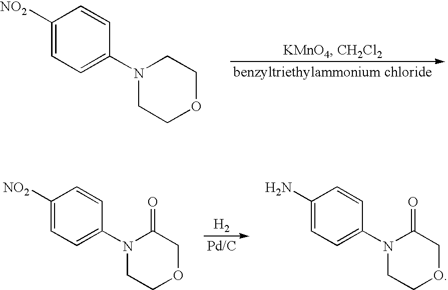

### 14.15 1-(4-Aminophenyl)pyridin-2-one

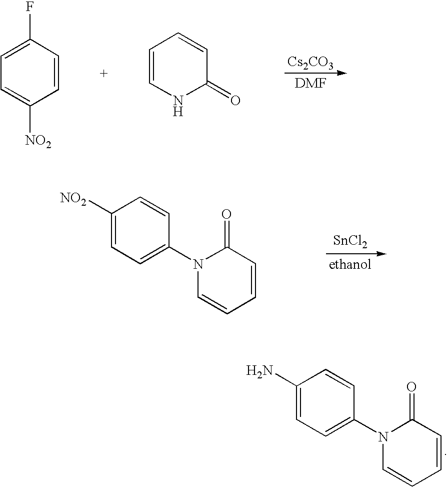

### 14.16 1-(4-Amino-2-methylphenyl)piperidin-2-one

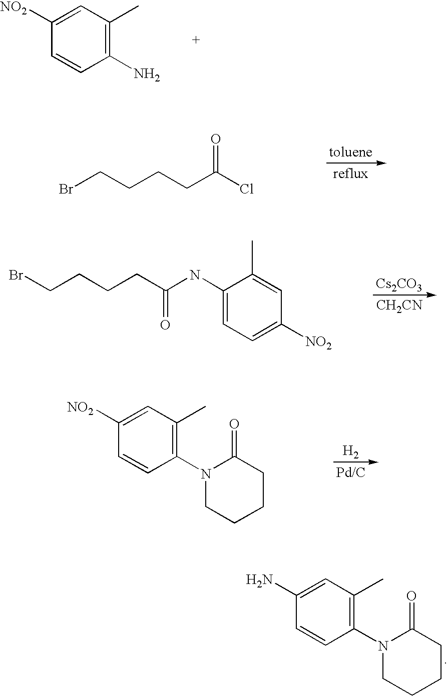

### 14.17 1-(4-Aminophenyl)-1H-pyridin-4-one

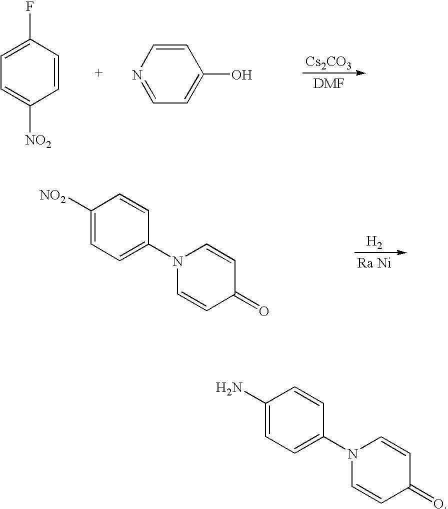

### 14.18 1-(4-Aminophenyl)-4-tert-butyloxycarbonylpiperazin-2-one

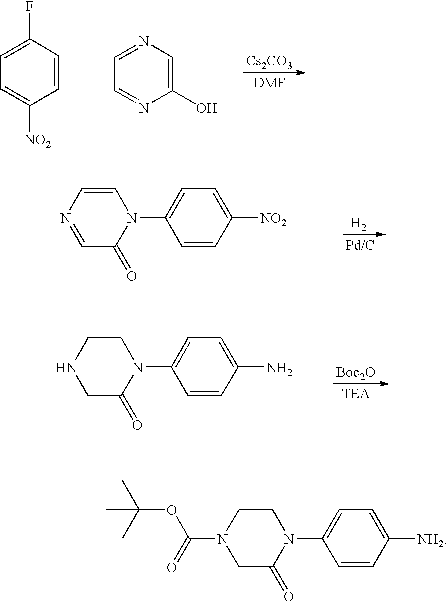

### 14.19 1-(3-Aminophenyl)piperidin-2-one

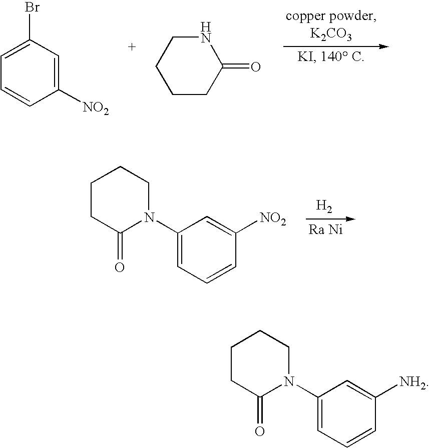

### 14.20 1-(4-Aminophenyl)-2-caprolactam

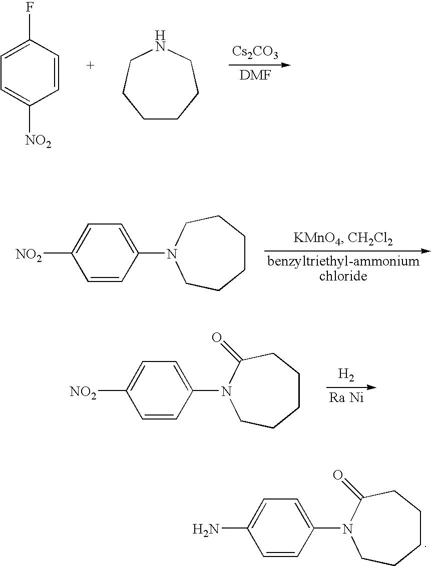

### 14.21 1-(4-Amino-3-fluorophenyl)piperidin-2-one

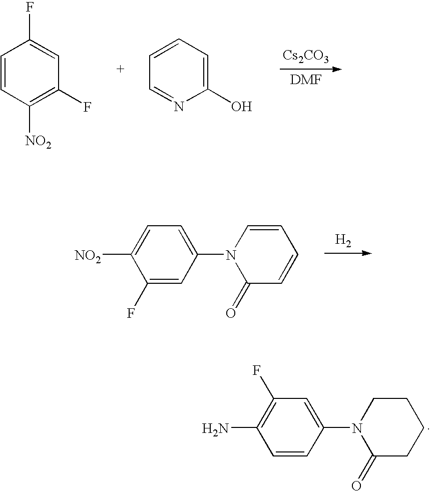

### 14.22 1-(4-Amino-2-fluorophenyl)piperidin-2-one

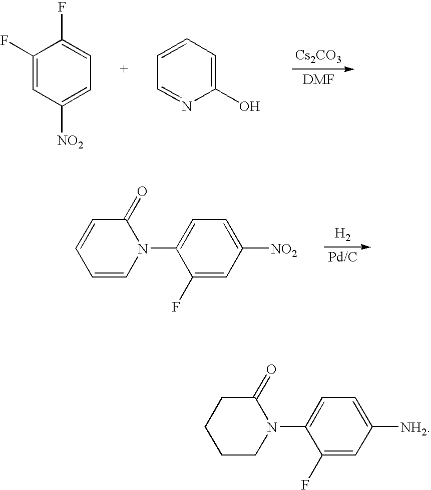

### 14.23 1-(4-Amino-2-fluorophenyl)-2-caprolactam

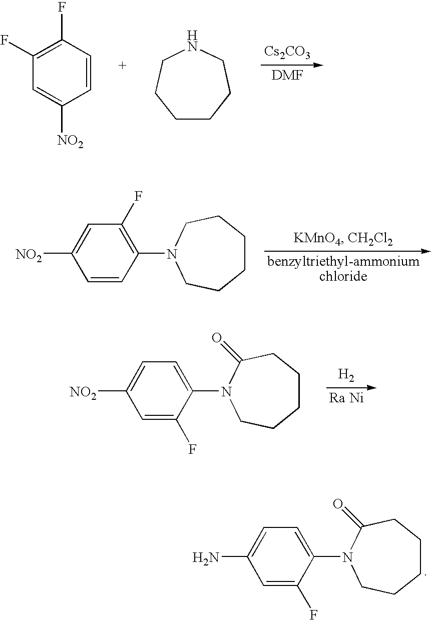

### 14.24 4-(4-Amino-2-fluorophenyl)-1,4-oxazepan-5-one

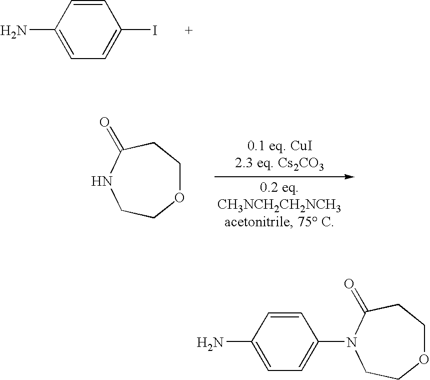

### 14.25 4-(4-Amino-3-phenoxyphenyl)morpholin-3-one

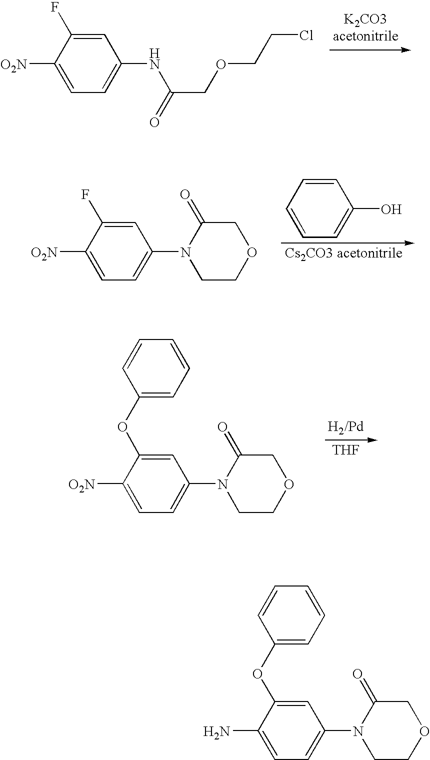

### 14.26 2-[3-(4-Chlorophenyl)ureido]cyclopentanecarboxylic acid

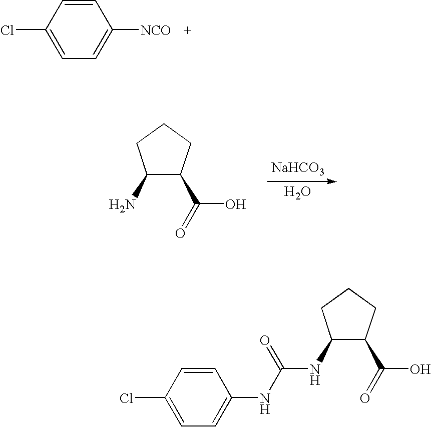

### 14.27 1-(4-Chlorophenylcarbamoyl)piperidine-3-carboxylic acid

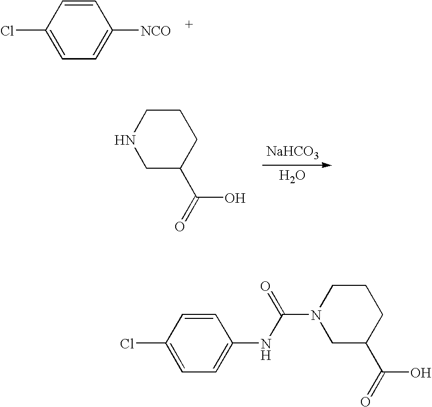

### 41 14.28 4-(4-Aminophenyl)-1,4-oxazepan-3-one

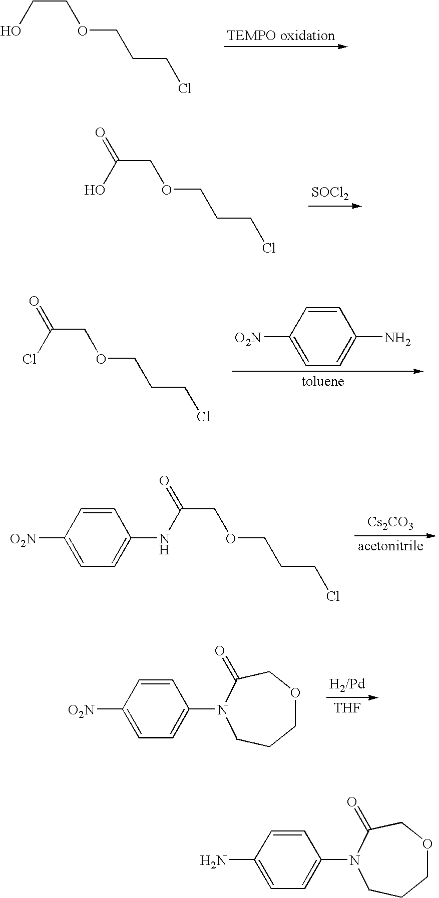

The TEMPO oxidation is carried out in accordance with the following lit-erature:

L. DeLuca et al., J. Org. Chem. 68, 4999-5001 (2003).

The following examples relate to pharmaceutical compositions:

### Example A

### Injection Vials

A solution of 100 g of an active ingredient of the formula I and 5 g of disodium hydrogenphosphate in 3 l of bidistilled water is adjusted to pH 6.5 using 2 N hydrochloric acid, sterile filtered, transferred into injection vials, lyophilised under sterile conditions and sealed under sterile conditions. Each injection vial contains 5 mg of active ingredient.

### Example B

### Suppositories

A mixture of 20 g of an active ingredient of the formula I with 100 g of soya lecithin and 1400 g of cocoa butter is melted, poured into moulds and allowed to cool. Each suppository contains 20 mg of active ingredient.

### Example C

### Solution

A solution is prepared from 1 g of an active ingredient of the formula I, 9.38 g of NaH₂PO₄.2H₂O, 28.48 g of Na₂HPO₄.12H₂O and 0.1 g of benzalkonium chloride in 940 ml of bidistilled water. The pH is adjusted to 6.8, and the solution is made up to 1 l and sterilised by irradiation. This solution can be used in the form of eye drops.

### Example D

### Ointment

500 mg of an active ingredient of the formula I are mixed with 99.5 g of Vaseline under aseptic conditions.

### Example E

### Tablets

A mixture of 1 kg of active ingredient of the formula I, 4 kg of lactose, 1.2 kg of potato starch, 0.2 kg of talc and 0.1 kg of magnesium stearate is pressed to give tablets in a conventional manner in such a way that each tablet contains 10 mg of active ingredient.

### Example F

### Coated Tablets

Tablets are pressed analogously to Example E and subsequently coated in a conventional manner with a coating of sucrose, potato starch, talc, tragacanth and dye.

### Example G

### Capsules

2 kg of active ingredient of the formula I are introduced into hard gelatine capsules in a conventional manner in such a way that each capsule contains 20 mg of the active ingredient.

### Example H

### Ampoules

A solution of 1 kg of active ingredient of the formula I in 60 l of bidistilled water is sterile filtered, transferred into ampoules, lyophilised under sterile conditions and sealed under sterile conditions. Each ampoule contains 10 mg of active ingredient.

1. Compounds of the formula I 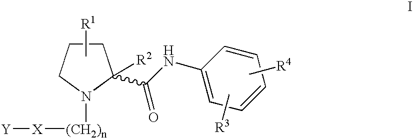
in which
R¹ denotes H, ═O, Hal, A, OH, OA, —O—(CH₂)ₘ—OA, A-COO—, Ph-(CH₂)ₙ—COO—, cycloalkyl-(CH₂)ₙ—COO—, A-CONH—, A-CONA-, Ph-CONA-, N₃, NH₂, NO₂, CN, COOH, COOA, CONH₂, CONHA, CON(A)₂, O-allyl,
O-propargyl, O-benzyl, ═N—OH, ═N-OA or ═CF₂,
R² denotes H or A,
Ph denotes phenyl which is unsubstituted or mono-, di- or trisubstituted by A, OA, OH and/or Hal,
R³ denotes H, Hal or A,
R⁴ denotes 2-oxopiperidin-1-yl, 2-oxopyrrolidin-1-yl, 2-oxo-1H-pyridin-1-yl, 3-oxomorpholin-4-yl, 4-oxo-1H-pyridin-1-yl, 2-oxo-1H-pyrazin-1-yl, 2-oxoimidazolidin-1-yl, 2-iminopiperidin-1-yl, 2-iminopyrrolidin-1-yl, 3-iminomorpholin-4-yl, 2-iminoimidazolidin-1-yl, 2-imino-1H-pyrazin-1-yl, 2,6-di-oxopiperidin 1-yl, 2-oxopiperazin-1-yl, 2,6-dioxopiperazin-1-yl, 2,5-dioxopyrrolidin-1-yl, 2-oxo-1,3-oxazolidin-3-yl, 3-oxo-2H-pyridazin-2-yl, 2-caprolactam-1-yl (=2-oxoazepan-1-yl), 2-azabicyclo[2.2.2]-octan-3-on-2-yl, 5,6-dihydro-1H-pyrimidin-2-oxo-1-yl, 2-oxo-1,3-oxazinan-3-yl or 4H-1,4-oxazin-4-yl, optionally mono- or disubstituted by A, OA, OH and/or CN,
X denotes a bond, CONH or NHCO,
Y denotes phenyl, pyridyl, thienyl, pyrimidyl, benzo[b]thiophenyl, each of which is unsubstituted or mono-, di-, tri-, tetra- or pentasubstituted by Hal,
or Z,
Z denotes 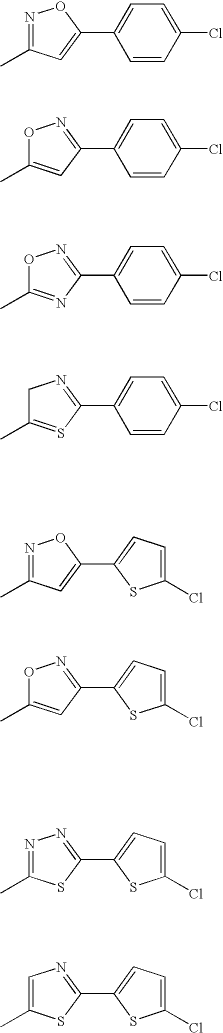
A denotes unbranched, branched or cyclic alkyl having 1-10 C atoms, in which, in addition, 1-7H atoms may be replaced by F and/or chlorine,
Hal denotes F, Cl, Br or I,
n denotes 1 or 2,
and pharmaceutically usable derivatives, solvates, salts and stereoisomers thereof, including mixtures thereof in all ratios.

2. Compounds according to claim 1 in which
R⁴ denotes 3-oxomorpholin-4-yl,
and pharmaceutically usable derivatives, solvates, salts and stereoisomers thereof, including mixtures thereof in all ratios.

3. Compounds according to claim 1 in which
R¹ denotes H,
R² denotes H.
R³ denotes H,
R⁴ denotes 3-oxomorpholin-4-yl,
and pharmaceutically usable derivatives, solvates, salts and stereoisomers thereof, including mixtures thereof in all ratios.

4. Compounds according to claim 1 selected from the group
1-N-[(5-chlorothiophen-2-yl)aminocarbonylmethyl]-2-N-[4-(3-oxomorpholin-4-yl)phenyl]-(R)-pyrrolidine-2-carboxamide,
1-N-[(5-chlorothiophen-2-yl)aminocarbonylmethyl]-2-N-[4-(3-oxomorpholin-4-yl)phenyl]-(S)-pyrrolidine-2-carboxamide,
1-N-[(4-chlorophenyl)aminocarbonylmethyl]-2-N-[4-(3-oxomorpholin-4-yl)phenyl]-(R)-pyrrolidine-2-carboxamide,
1-N-[(3-chloropyridin-6-yl)aminocarbonylmethyl]-2-N-[4-(3-oxomorpholin-4-yl)phenyl]-(R)-pyrrolidine-2-carboxamide,
1-N-[2-(4-chlorophenyl)ethyl]-2-N-[4-(3-oxomorpholin-4-yl)phenyl]-(R)-pyrrolidine-2-carboxamide,
1-N-[(6-chlorobenzo[b]thiophen-2-yl)methyl]-2-N-[4-(3-oxomorpholin-4-yl)phenyl]-(R)-pyrrolidine-2-carboxamide,
1-N-[(3-(4-chlorophenyl)isoxazol-5-yl)methyl]-2-N-[4-(3-oxo-morpholin-4-yl)phenyl]-(R)-pyrrolidine-2-carboxamide,
1-N-[(5-(4-chlorophenyl)isoxazol-3-yl)methyl]-2-N-[4-(3-oxo-morpholin-4-yl)phenyl]-(R)-pyrrolidine-2-carboxamide,
1-N-[(3-(4-chlorophenyl)-1,2,4-oxdiazol-5-yl)methyl]-2-N-[4-(3-oxo-morpholin-4-yl)phenyl]-(R)-pyrrolidine-2-carboxamide,
1-N-[(2-(4-chlorophenyl)thiazol-5-yl)methyl]-2-N-[4-(3-oxomorpholin-4-yl)phenyl]-(R)-pyrrolidine-2-carboxamide,
1-N-[(3-(2-chlorothiophen-5-yl)isoxazol-5-yl)methyl]-2-N-[4-(3-oxo-morpholin-4-yl)phenyl]-(R)-pyrrolidine-2-carboxamide,
1-N-[(5-(2-chlorothiophen-5-yl)isoxazol-3-yl)methyl]-2-N-[4-(3-oxo-morpholin-4-yl)phenyl]-(R)-pyrrolidine-2-carboxamide,
1-N-[(2-(2-chlorothiophen-5-yl)-1,3,4-thiadiazol-5-yl)methyl]-2-N-[4-(3-oxomorpholin-4-yl)phenyl]-(R)-pyrrolidine-2-carboxamide,
1-N-[(2-(2-chlorothiophen-5-yl)thiazol-5-yl)methyl]-2-N-[4-(3-oxo-morpholin-4-yl)phenyl]-(R)-pyrrolidine-2-carboxamide,
and pharmaceutically usable derivatives, solvates, salts and stereoisomers thereof, including mixtures thereof in all ratios.

5. Process for the preparation of compounds of the formula I according to claim 1 and pharmaceutically usable derivatives, solvates, salts and stereoisomers thereof, characterised in that a compound of the formula II 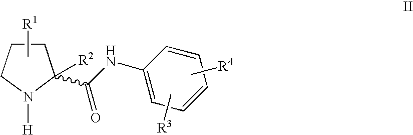
in which R¹, R², R³ and R⁴ have the meanings indicated in claim 1,
is reacted with a compound of the formula III
Y—X—(CH₂)ₙ-L III
in which
L denotes Cl, Br, I or a free or reactively functionally modified OH group and
X, Y and n have the meanings indicated in claim 1,
and/or
a base or acid of the formula I is converted into one of its salts.

6. Compounds of the formula I according to claim 1 as inhibitors of coagulation factor Xa.

7. Compounds of the formula I according to claim 1 as inhibitors of coagulation factor VIIa.

8. Medicaments comprising at least one compound of the formula I according to one or more of claims 1 to 4 and/or pharmaceutically usable derivatives, solvates, salts and stereoisomers thereof, including mixtures thereof in all ratios, and optionally excipients and/or adjuvants.

9. Medicaments comprising at least one compound of the formula I according claim 1 and/or pharmaceutically usable derivatives, solvates and stereoisomers thereof, including mixtures thereof in all ratios, and at least one further medicament active ingredient.

10. Use of compounds according to claim 1 and/or physiologically acceptable salts, salts and solvates thereof for the preparation of a medicament for the treatment of thromboses, myocardial infarction, arteriosclerosis, inflammation, apoplexy, angina pectoris, restenosis after angioplasty, claudicatio intermittens, migraine, tumours, tumour diseases and/or tumour metastases.

11. Use of compounds according to claim 1 and/or physiologically acceptable salts, salts and solvates thereof for the preparation of a medicament for the prevention and treatment of thromboembolic diseases and/or thromboses as a consequence of a surgical intervention, genetically caused diseases having increased thrombosis suitability, diseases of the arterial and venous vascular system, cardiac insufficiency, atrial fibrillation, thrombophilia, tinnitus and/or sepsis.

12. Use according to claim 11, where the surgical interventions are selected from the group thorax operations, operations in the abdominal region, orthopaedic interventions, hip and knee joint replacement, CABG (coronary artery bypass grafting), artificial heart-valve replacement, operations with use of a heart-lung machine, vascular surgery, organ transplants and use of central vein catheters.

13. Set (kit) consisting of separate packs of
(a) an effective amount of a compound of the formula I according to claim 1 and/or pharmaceutically usable derivatives, solvates, salts and stereoisomers thereof, including mixtures thereof in all ratios,
and
(b) an effective amount of a further medicament active ingredient.

14. Use of compounds of the formula I according to claim 1 and/or pharmaceutically usable derivatives, solvates, salts and stereoisomers thereof, including mixtures thereof in all ratios,
for the preparation of a medicament for the treatment of thromboses, myocardial infarction, arteriosclerosis, inflammation, apoplexy, angina pectoris, restenosis after angioplasty, claudicatio intermittens, migraine, tumours, tumour diseases and/or tumour metastases,
for the prevention and treatment of thromboembolic diseases and/or thromboses as a consequence of a surgical intervention, genetically caused diseases having increased thrombosis suitability, diseases of the arterial and venous vascular system, cardiac insufficiency, atrial fibrillation, thrombophilia, tinnitus and/or sepsis, in combination with at least one further medicament active ingredient.
